(19) United States
(12) Patent Application Publication
(10) Pub. No.: US 2020/0200748 A1
(43) Pub. Date: Jun. 25, 2020

(54) METHOD FOR DETECTING AGGREGATES OF BIOTHERAPEUTIC SUBSTANCES IN A SAMPLE

(71) Applicant: FORSCHUNGSZENTRUM JUELICH GMBH, JUELICH (DE)

(72) Inventors: Christian Zafiu, Vienna (AU); Andreas Kulawik, Erkrath (DE); Oliver Bannach, Duesseldorf (DE); Dieter Willbold, Juelich (DE)

(21) Appl. No.: 16/612,769

(22) PCT Filed: May 15, 2018
(86) PCT No.: PCT/DE2018/000139

§ 371 Date: Nov. 12, 2019

(30) Foreign Application Priority Data
Jun. 13, 2017 (DE) 10 2017 005 544.0
Nov. 13, 2017 (DE) 10 2017 010 455.7

(51) Int. Cl. G01N 33/552; G01N 33/545; G01N 33/58; G01N 33/68; G01N 33/543; G01N 21/64

(52) U.S. Cl. CPC G01N 33/552; G01N 33/545; G01N 33/582; G01N 2021/7786; G01N 33/54393; G01N 33/54353; G01N 21/6458; G01N 33/6803

(57) ABSTRACT

The invention relates to a method for detecting aggregates of biotherapeutic substances in a sample, said method involving the following steps: a) applying the sample to be examined to a substrate; b) adding probes which are labeled for the detection and which mark the aggregates of biotherapeutic substances by specifically binding thereto; and c) detecting the labeled aggregates of the biotherapeutic substances wherein step a) can be carried out prior to step b). A kit for carrying out said method is also disclosed.

### CROSS REFERENCE TO PRIOR APPLICATIONS

This application is a U.S. National Phase application under 35 U.S.C. § 371 of International Application No. PCT/DE2018/000139, filed on May 15, 2018, and claims benefit to German Patent Application No. 10 2017 005 544.0, filed on Jun. 13, 2017, and German Patent Application No. 10 2017 010 455.7, filed on Nov. 13, 2017. The International Application was published in German on Dec. 20, 2018 as WO 2018/228622 A1 under PCT Article 21(2).

### FIELD

The invention relates to a method for detecting aggregates of biotherapeutic substances in a sample.

### BACKGROUND

Biopharmaceutical products are classified into biological products and their imitator preparations, biosimilars, and are the category of therapeutics produced in living organisms. These products include, but are not limited to, recombinant proteins and antibodies. These products play a key role in the treatment of various diseases, such as diabetes, various types of cancer and inflammatory diseases.

Biopharmaceutical products are highly attractive therapeutics from a medical point of view since proteins and antibodies have outstanding activities and specificities with regard to their effect. However, due to the structural complexity of these high-molecular substances in comparison to classical low-molecular pharmaceuticals, substantial challenges are found in the area of physical and chemical stability. Misfolding of such proteins, and in further consequence, aggregation, can take place during each individual phase of the product cycle of such a therapeutic. These stages include expression, folding, purification, sterilization, shipment, storage and delivery of the product.

The consequences of protein aggregation are firstly a reduction in activity and, particularly disadvantageously, an increased immunogenicity of the product. If the immune system recognizes the active ingredient as an antigen and forms antibodies against it, the result is the decomposition of the active ingredient or even an allergic reaction. This makes the treatment ineffective, possibly even dangerous.

As homogeneous protein aggregates are defined any self-associated protein species, with the monomer representing the smallest naturally occurring unit or subunit. Aggregates are classified according to the five characteristics size, reversibility (dissociation), conformation, chemical modification, and morphology [1].

The smallest aggregate unit corresponds to two monomers (dimer), no upper limit of the number of monomers subsequently being set [1]. Disadvantageously, immune responses have already been found even for the smallest aggregate units of biopharmaceutical protein products [2].

In addition to homogeneous protein aggregates, there are also heterogeneous aggregates in which the protein can associate monomers with one or more other protein species or even organic or inorganic impurities.

The underlying mechanisms which can elicit or intensify an immune response as a result of aggregates vary. Said mechanisms include a) increased cross-linking of B-cell receptors, which can cause their activation [3], b) increased antigen uptake, processing and presentation with subsequent triggering of immunostimulatory signals [4]. Such mechanisms can excite T-cells to produce antibodies. The greatest clinical danger of an immune response caused by aggregates depends on the preservation or degeneration of monomer epitopes in the aggregate. In the case of preservation of the epitopes, antibodies which were originally only directed against the aggregate can also bind monomers and reduce or neutralize their effect. In the case of degeneration, the antibodies are exclusively directed against aggregates, while the active substance activity of the native protein is not impaired. In both cases, the immune response may lead right up to anaphylaxis which may be dangerous to the patient.

Currently, there is no standardized method for analyzing impurities, the aggregate fraction and its size in biopharmaceutical preparations. Aggregates are classified by size into two categories, and suitable measurement methods are proposed.

Aggregates >1 μm: Optical methods are typically used for the determination of large aggregates and impurities, such as light attenuation (LO), dynamic imaging particle analysis (DIPA) and micro-flow imaging (MFI), as well as the electrochemical method of the Coulter counter (CC). With mature forms of these methods, it is also possible to determine the number, size and shape of the aggregates present.

Aggregates between 0.1 μm and 1 μm: Complex systems for the detection of small aggregates are used in this size range. These systems include size exclusion chromatography (SEC), analytical ultracentrifugation (AUC) and asymmetric flow field flow fractionation (AF4). In order to increase the sensitivity of such devices, they are often coupled to a mass spectrometer. Indeed, these techniques allow the quantification and distribution of aggregates. However, it is disadvantageous to determine the composition, and these methods are suitable only to a limited extent for high throughput applications.

Information about the type of aggregate and the quantity of aggregates is necessary in order to determine from which aggregate fraction an immune response to the therapeutic protein takes place [8]. Until now, the immune response was attributed mainly to large aggregates and particles [3], even though, without being bound by a particular theory, it is conceivable that the aggregates and amounts formed can vary from product to product and can lead to various clinical scenarios.

Although the finding that even particles and aggregates in the range of 0.1-10 μm can potentially have an immunogenic effect is slowly becoming accepted, the currently used methods lack the precision to determine it [5].

### SUMMARY

A method for detecting aggregates of biotherapeutic substances in a sample which includes (a) applying the sample to be examined onto a substrate, (b) adding probe molecules which are suitable for detection and which mark the aggregates of biotherapeutic substances by specifically binding thereto, and (c) detecting the marked aggregates of biotherapeutic substances, wherein step (b) may be carried out before step (a).

### BRIEF DESCRIPTION OF THE DRAWINGS

FIG. 1 shows aggregates (shaded bars) and monomers (white bars) of the human IgG antibody (isotype control, ThermoFisher Scientific, RF237824) in a decadic dilution series.

### DETAILED DESCRIPTION

In an embodiment, the present invention provides a highly sensitive method for detecting aggregates of biotherapeutic substances in a sample.

In an embodiment, the present invention provides a kit for carrying out the method.

Further embodiments arise from the description of the invention and the dependent claims.

An embodiment of the present invention is a method for detecting aggregates of biotherapeutic substances in a sample, comprising the following steps:

a. applying the sample to be examined onto a substrate,

b. adding probes which are suitable for detection and which mark the aggregates of biotherapeutic substances by specifically binding thereto, and

c. detecting the marked aggregates of biotherapeutic substances, wherein step b) may be carried out before step a).

In one embodiment, it is thus also possible to first add the probe and then the sample to the substrate.

The method for detecting and in particular for quantitatively and/or qualitatively determining homogeneous and heterogeneous aggregates is characterized in that aggregates of and in biopharmaceutical products contain at least one binding site for a probe.

Optionally, the aggregate also comprises at least one binding site for a capture molecule.

In one embodiment, the method comprises the following steps:

a) immobilizing capture molecules on a substrate,

b) bringing the sample into contact with a biopharmaceutical product in solution with the capture molecules,

c) immobilizing monomers and/or aggregates of one or more molecules present in solution of a biopharmaceutical product on the substrate by binding to the capture molecules,

d) bringing a probe into contact with the monomers and/or aggregates,

e) binding the probe to the monomers and/or aggregates,

wherein the probe is able to generate a defined signal and steps b) and d) can take place simultaneously or step d) can take place before step b).

If steps b) and d) occur simultaneously, steps c) and e) are thus also carried out simultaneously.

In a further embodiment, in which step d) is carried out before step b), immobilizing monomers and aggregates marked with probes on the substrate is thus carried out in step c). Consequently, step e) also takes place before steps b) and c).

For the purposes of the present invention, “quantitative determination” first means the determination of the concentration of the aggregates and thus also the determination of their presence and/or absence.

Preferably, “quantitative determination” also means the selective quantification of aggregate compositions. Such a quantification can take place via the corresponding probes.

For the purposes of the present invention, “qualitative determination” means characterization of the aggregate composition.

The aggregates are marked with one or more probes serving for detection. The probes contain an affinity molecule which recognizes and binds to a binding site of the aggregate or its monomer.

Moreover, the probes contain at least one detection molecule or a molecular moiety which is bound to the affinity molecule or molecular moiety and can be detected or measured by means of chemical or physical methods.

In one embodiment, the probes may have identical affinity molecules or molecular moieties with different detection molecules (or moieties). In another embodiment, different affinity molecules or molecular moieties may be combined with different detection molecules or moieties, or, alternatively, different affinity molecules or moieties may be combined with identical detection molecules or moieties. It is also possible to use mixtures of various probes.

The use of a plurality of different probes coupled to different detection molecules or molecular moieties can increase the specificity of the signal (correlation signal) on the one hand and this allows on the other hand the identification of aggregates which differ in their composition. This enables selective quantification and characterization of the aggregates.

In one embodiment, a spatially resolved determination of the probe signal, that is to say a spatially resolved detection of the signal emitted by the probe, takes place. Accordingly, in this embodiment of the invention, methods based on a non-spatially resolved signal, such as ELISA or sandwich ELISA, are excluded.

High spatial resolution is advantageous but not essential in the detection. In one embodiment of the method according to the invention, enough data points are collected that the detection of an aggregate in front of a background signal, which is caused, for example, by device-specific noise, other unspecific signals or non-specifically bound probes, is made possible. In this way, as many values (read-out values) are read out as there are spatially resolved events, such as pixels. The spatial resolution determines each event in front of the respective background and thus constitutes an advantage over ELISA methods without a spatially resolved signal.

In one embodiment, the spatially resolved determination of the probe signal is based on the examination of a small volume element in comparison to the volume of the sample, in the range from a few femtoliters to below one femtoliter, or of a volume range above the contact surface of the capture molecules with a height of 500 nm, preferably 300 nm, particularly preferably 250 nm, in particular 200 nm, but also 150 nm and 90 nm.

In the context of the invention, aggregates are either homogeneous aggregates consisting of at least two identical monomer units or heterogeneous aggregates consisting of at least two different monomer units. In the case of heterogeneous aggregates, both monomers may also be identical in their primary sequence but differ in their conformation.

In one embodiment, the material of the substrate is selected from the group comprising or consisting of plastic, silicon and silicon dioxide. In a preferred alternative, glass is used as the substrate.

In a further embodiment of the invention, the capture molecules are covalently bound to the substrate.

In another embodiment, a substrate having a hydrophilic surface is used for this purpose. In a further embodiment, this is achieved by applying a hydrophilic layer onto the substrate prior to step a). Consequently, the capture molecules covalently bind to the substrate or to the hydrophilic layer with which the substrate is loaded.

The hydrophilic layer is a biomolecule-repellent layer so that non-specific binding of biomolecules to the substrate is minimized. The capture molecules are optionally immobilized, preferably covalently, onto this layer. These capture molecules have affinity to a feature of the monomers or their aggregates. The capture molecules may all be identical or be mixtures of various capture molecules.

In an alternative, the same molecules are used as capture molecules and probes; the capture molecules preferably contain no detection molecule or molecular moieties.

In one embodiment, the hydrophilic layer is selected from the group comprising or consisting of PEG, polylysine, preferably poly-D-lysine, and dextran or derivatives thereof, preferably carboxymethyl-dextran (CMD). Derivatives within the meaning of the invention are compounds which differ in some substituents from the parent compounds, the substituents being inert to the method embodiments according to the invention.

In one embodiment, the surface of the substrate is first hydroxylated before application of the hydrophilic layer and is subsequently functionalized with suitable chemical groups, preferably amino groups. This functionalization with amino groups is carried out in an alternative by bringing the substrate into contact with aminosilanes, preferably APTES (3-aminopropyltrietoxysilane), or with ethanolamine.

In certain embodiments, in order to prepare the substrate for the coating, one or more of the following steps can be carried out:
washing a substrate of glass or a glass carrier in an ultrasonic bath or plasma cleaner; alternatively, incubating in 5 M NaOH for at least 3 hours,
rinsing with water and subsequently drying under nitrogen or under vacuum,
dipping into a solution of concentrated sulfuric acid and hydrogen peroxide at a ratio of 3:1 for the activation of the hydroxyl groups,
rinsing with water to a neutral pH, subsequently washing with ethanol and drying under a nitrogen atmosphere,
dipping into a solution of 3-aminopropyltrietoxysilane (APTES) (1-7%), preferably in dry toluene, or a solution of ethanolamine,
rinsing with acetone or DMSO and water, and drying under a nitrogen atmosphere.

In a further embodiment, the substrate is brought into contact with aminosilanes, preferably APTES, in the gas phase; the optionally pretreated substrate is consequently vaporized with the aminosilanes.

For coating with dextran, preferably carboxymethyl-dextran (CMD), the substrate is incubated with an aqueous solution of CMD (at a concentration of 10 mg/ml or 20 mg/ml) and with a catalyst for covalent coupling, optionally N-ethyl-N-(3-dimethylaminopropyl)carbodiimide (EDC) (200 mM) and N-hydroxysuccinimide (NHS) (50 mM), and subsequently washed.

In one embodiment, the carboxymethyl-dextran in one variant is covalently bound to the glass surface, which was first hydroxylated and subsequently functionalized with amine groups, as described above.

Microtiter plates, preferably with a glass bottom, can be used as the substrate. Since the use of concentrated sulfuric acid is not possible when polystyrene frames are used, the glass surface is activated analogously in an embodiment variant of the invention.

Capture molecules are immobilized, preferably covalently, onto this hydrophilic layer, said molecules having affinity to a feature (e.g.: proteins) of the aggregate. The capture molecules may all be identical or be mixtures of various capture molecules.

In one embodiment of the present invention, the capture molecules, preferably antibodies to monomers of the aggregate, are optionally immobilized on the substrate by a mixture of EDC/NHS, preferably 200 and 50 mM respectively, after activation of the CMD-coated carrier.

Remaining carboxylate end groups to which no capture molecules were bound can be deactivated. Ethanolamine is used to deactivate these carboxylate end groups on the CMD spacer. Prior to the application of the samples, the substrates or carriers are optionally rinsed with buffer.

In one embodiment of the present invention, the substrate is blocked with protein or peptide-containing solutions prior to application of the sample and washed with buffer.

In certain embodiments, the sample to be measured is brought into contact with the substrate prepared in this way and optionally incubated. Differently formulated solutions of the biopharmaceutical product, of the product in cell supernatants and culture media or endogenous fluids can be used as the sample to be examined. In one embodiment of the present invention, the sample is selected from liquor (CSF), blood, plasma and urine. The samples may undergo various processing steps known to the person skilled in the art.

In one embodiment of the present invention, the sample is applied directly onto the substrate (non-coated substrate), optionally by covalent binding to the optionally activated surface of the substrate.

In one variant of the present invention, the sample is pretreated according to one or more of the following methods:
heating of the sample,
one or more freeze-thawing cycles,
mechanical digestion,
homogenization of the sample,
diluting with water or buffer,
treating with enzymes, for example nuclease, lipases,
centrifuging,
competition with probes in order to displace any antibodies present.

Preferably, the sample is brought into contact with the substrate directly and/or without pretreatment.

Non-specifically bound substances can be removed by washing steps.

In a further step, immobilized aggregates are marked with one or more probes serving for further detection. As described above, the individual steps can also be performed in a different order according to the invention.

By suitable washing steps, excess probes which are not bound to the aggregates are removed.

In one embodiment, these excess probes are not removed. As a result, the last washing steps are omitted and there is also no equilibrium shift in the direction of dissociation of the aggregate-probe complexes or compounds. By means of the spatially resolved detection, the excess probes are not detected during the analysis and do not impair the measurement.

In one embodiment, the sample-capture molecule complexes are chemically fixed.

In another embodiment, detection probe-sample-capture molecule complexes are chemically fixed and thus also the sample-capture molecule complexes.

In a particular embodiment of the method, the binding sites of the aggregate epitopes and the capture molecules and probes are antibodies. In one embodiment of the present invention, capture molecules and probes may be identical.

In one embodiment of the present invention, capture molecules and probes differ. For example, various antibodies can be used as capture molecules and as probes.

In a further embodiment of the present invention, capture molecules and probes which are identical to one another with the exception of the possible (dye) marking are used.

In a further embodiment of the present invention, various probes which are identical to one another with the exception of the possible (dye) marking are used.

In further embodiments of the present invention, at least two or more different capture molecules and/or probes which contain different antibodies and optionally also have different dye markings are used.

In one embodiment of the invention, two or more probes are marked with corresponding dyes in such a way that a FRET, a so-called Förster resonant energy transfer, takes place, wherein one dye is excited and another dye in the vicinity is emitted, wherein both dyes are different molecules.

In certain embodiments, for detection purposes, the probes are marked such that they emit an optically detectable signal selected from the group consisting of fluorescence, phosphorescence, bioluminescence, chemiluminescence and electrochemiluminescence emission as well as absorption.

In certain embodiments, the probes are thus marked with fluorescent dyes. The dyes known to the person skilled in the art can be used as fluorescent dye. Alternatively, fluorescent biomolecules, preferably GFP (green fluorescent protein), conjugates and/or fusion proteins thereof, as well as fluorescent nanoparticles, preferably quantum dots, can also be used.

Catcher molecules marked with fluorescent dyes can be used for the later quality control of the surface, for example the evenness of the coating with capture molecules. A dye which does not interfere with the detection is preferably used for this purpose. This enables subsequent control of the structure and standardization of the measurement results.

The immobilized and marked aggregates are detected by imaging the surface (e.g., laser microscopy). As high a spatial resolution as possible determines a high number of image points, as a result of which the sensitivity and the selectivity of the assay can be increased since structural features can also be imaged and analyzed. Thus, the specific signal in front of the background signal (e.g., non-specifically bound probes) increases.

In certain embodiments, detection preferably takes place with confocal fluorescence microscopy, fluorescence correlation spectroscopy (FCS), in particular in combination with cross-correlation and laser scanning microscopy (LSM).

In an embodiment of the present invention, the detection is carried out with a confocal laser scanning microscope.

In one embodiment of the present invention, a laser focus, such as is used in laser scanning microscopy, or an FCS (fluorescence correlation spectroscopy system) is used for this purpose as well as the corresponding super-resolution variants, such as STED, PALM or SIM.

In a further embodiment, the detection can be effected by means of spatially resolving fluorescence microscopy, preferably by a TIRF microscope, and the corresponding super-resolution variants thereof, such as STORM, dSTORM.

In contrast to ELISA, these methods result in as many read-out values as there are spatially resolved events (e.g., pixels). Depending on the number of different probes, this information is even multiplied. This multiplication applies to each detection event and leads to information gain since it discloses further properties (e.g., second feature) of aggregates. As a result of such a structure, the specificity of the signal can be increased for each event.

The probes can be selected in such a way that, in the case of heterogeneous aggregates, the presence of individual constituents or conformations does not influence the measurement result. The probes can be selected in such a way that homogeneous and heterogeneous aggregates and various heterogeneous aggregates can be determined in one measurement.

For analysis, the spatially resolved information (e.g., the fluorescence intensity) of all probes used and detected is used in order to determine, for example, the number of aggregates, their size and their features. For example, algorithms for background minimization and/or intensity threshold values can also be used for further analysis as well as pattern recognition. Further image analysis options include, for example, the search for local intensity maxima in order to obtain from the image information the number of aggregates detected.

In order to make the test results comparable with one another across distances, times and experimenters, standards (controls), for example internal and/or external standards (controls), can be used. These standards (controls) can also serve to calibrate the measurement in order to determine the size distribution, quantity and/or composition of aggregates of biopharmaceutical products.

Certain embodiments of the present invention provide standards (controls) which have a narrow size distribution and which consist of two or more identical or different polypeptide sequences. The polypeptide sequences may also be the native monomeric form of the biopharmaceutical agent.

In one embodiment, the standard (control) consists of a mixture of various size distributions.

In one embodiment, the standard (control) is marked.

In one embodiment, the standard (control) consists of two or more monomers covalently linked to one another.

In one embodiment, the standard (control) consists of a nanoparticle to the surface of which two or more monomers or a polypeptide sequence are covalently bound, and the polypeptide sequence is identical in a partial region with the sequence of the monomer of the biotherapeutic substances.

Another embodiment of the present invention is a kit containing one or more of the following components:

substrate, optionally with hydrophilic surface, capture molecule, probe, standard, substrate with capture molecule, solutions, buffer.

In one embodiment, the compounds and/or components of the kit of the present invention may be packaged in containers, optionally with/in buffers and/or solution. In another embodiment, some components may be packaged in the same container. In a further embodiment, one or more of the components could be adsorbed to a solid carrier, such as a glass plate, a chip or a nylon membrane, or to the well of a microtiter plate. In yet a further embodiment, the kit may furthermore include instructions for use of the kit for any of the embodiments.

In a further embodiment, the capture molecules described above are immobilized on the substrate. In addition, the kit may contain solutions and/or buffers. In order to protect the biomolecule-repellent surface (e.g., dextran surface) and/or the capture molecules immobilized thereon, they can be overlaid with a solution or a buffer. In an alternative embodiment, the solution contains one or more biocides which increase the shelf life of the surface.

Another embodiment of the present invention is the use of the method according to the invention for detecting homogeneous and heterogeneous aggregates of and in biopharmaceutical products in any samples, for quantifying (titer determination) homogeneous and heterogeneous aggregates of and in biopharmaceutical products, and for directly and/or absolutely quantifying the particle number.

Another embodiment of the present invention is the use of the method according to the invention for optimizing and monitoring process steps during the production of biopharmaceutical agents and/or for determining the quality of end products.

Another embodiment of the present invention is the use of the method according to the invention for detecting homogeneous and heterogeneous aggregates of biopharmaceutical products in clinical trials, studies and in treatment monitoring. For this purpose, samples are measured according to the method according to the invention and the results are compared.

### EXAMPLES

An exemplary embodiment and the appended FIGURE is described below, without this resulting in a restriction of the invention to this specific exemplary embodiment.

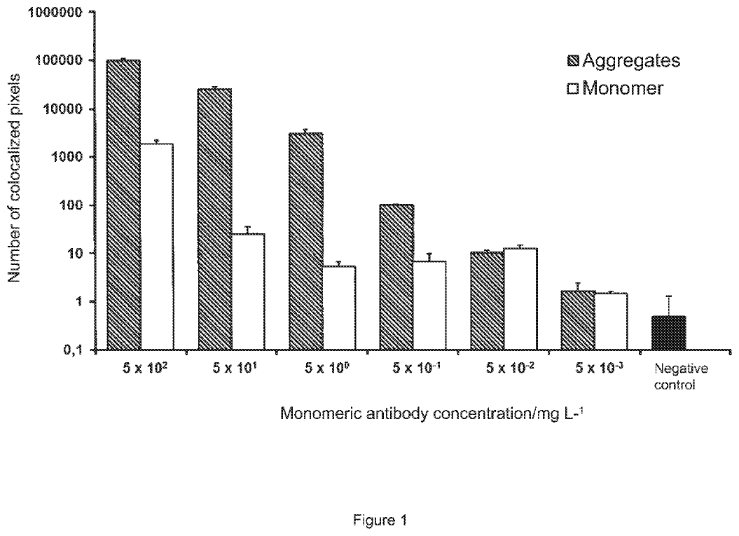

The FIGURES show:
FIG. 1 aggregates (shaded bars) and monomers (white bars) of the human IgG antibody as sample.
FIG. 1 shows aggregates (shaded bars) and monomers (white bars) of the human IgG antibody (isotype control, ThermoFisher Scientific, RF237824) in a decadic dilution series.

The antibody as presented was used as monomer and diluted in a saline phosphate buffer (PBS) at a pH of 7.4. For the preparation of aggregates, the sample (5 mg/ml) was heated for 10 min at 70° C. and decadically diluted after reaching 25° C.

The results show a linear relationship between the concentration and the measurement signal over 6 log stages. On the other hand, monomers exhibit far lower measurement signals over wide ranges. It cannot be ruled out that even the monomer solution contained small amounts of aggregate which were responsible for values at high concentrations. The dilution buffer was used as a negative control (black bar).

### Specific Embodiment

For the experiment, commercial microtiter plates (Greiner Bio-one; Sensoplate Plus) with 384 reaction chambers (RK) and glass bottom were used as substrate.

First, the surface of the microtiter plate was constructed or functionalized. For this purpose, the plate was placed into a desiccator in which a tray with 5% APTES in toluene was located. The desiccator was flooded with argon and incubated for one hour. The tray was then removed, and the plate was dried in vacuo for 2 hours. 20 μl of a 2 mM solution of SC-PEG-CM (MW 3400; Laysan Bio) in deionized H₂O for the hydrophilic coating were poured into the reaction chamber of the dry plate and incubated for 4 hours. After incubation, the reaction chamber was washed three times with water and subsequently incubated with 20 μl each of an aqueous 200 mM EDC solution (1-ethyl-3-(3-dimethylaminopropyl)carbodiimide; sigma) and with 50 mM NHS (N-hydroxysuccinimide, sigma) for 30 minutes. This gives the hydrophilic coating with PEG a functionality for coupling biomolecules.

The plate was again washed three times with deionized water. Thereafter, the reaction chamber was coated with Klon 8A4 (Thermo-Fischer), a monoclonal antibody as a capture molecule, which specifically binds the CH2 domain in the FC part of human antibodies (20 μl; 10 μg/ml in PBS; 1 hour). Afterwards, the reaction chamber was treated with the washing program consisting of three times washing and sucking empty with TBS with 0.1% Tween-20 and TBS.

In the next step, the reaction chamber was coated with 50 μl Smartblock (Candor Bioscience GmbH) overnight at room temperature (RT) and, after the time passed, again washed three times with saline tris(hydroxymethyl)aminomethane (TBS; pH=7.4).

The samples were sequentially diluted in tris(hydroxymethyl)aminomethane (TRIS) buffer with Hoechst Stain dye (1 μg ml−1) and incubated for one hour. Thereafter, three times, 20 μl each of the sample was applied to the reaction chamber and incubated at room temperature for 1 hour. After incubation, the reaction chamber was washed three times with TBS and 20 μl of detection antibodies were added. The detection antibodies were each marked with a type of fluorescent dye: 8A4 (ThermoFisher Scientific, MA1-81864) was marked separately with fluorescent dyes CF488 and CF633. These probe antibodies and detection antibodies were diluted together in TBS to a final concentration of 1.25 ng/ml for each antibody. They specifically bind epitopes of the monomers and aggregates of the biotherapeutic substance.

In each reaction chamber, 20 μl of antibody solution were applied and incubated for 1 hour at room temperature. After the time passed, the plate was washed three times with TBS and the plate was sealed with a film.

Spatially resolved microscopy was carried out in order to detect the aggregates. The measurement was carried out in the TIRF microscope (Leica) with a 100-fold oil immersion objective. For this purpose, the glass bottom of the microtiter plate was coated abundantly with immersion oil, and the plate was introduced into the automated stage of the microscope. One image (1000×100 pixels) each was then recorded consecutively per reaction chamber at 5×5 positions in two fluorescence channels (Ex/Em=633/715 nm and 488/525 nm) in order to obtain enough data points that the detection of individual aggregates in front of the background signal was made possible. The maximum laser power (100%), an exposure time of 500 ms and a gain value of 800 were selected. The image data were then analyzed. Intensity threshold values were set for each channel at 0.0001% gray levels of the average negative control in the corresponding channel. In the analysis step, the intensity threshold value was first applied for each image in each channel and images of the same position were subsequently compared with one another in both values. Only those pixels per image were counted in which, in both channels, the pixel is located at exactly the same position above the intensity threshold value of the channel. Lastly, the number of pixels over all images in each RK is averaged and the mean values of the average pixel numbers of the replica values are then determined and the standard deviation is specified.

The values are shown in FIG. 1.

This calibration series can then serve as an entry into more complex detection methods of biotherapeutic substances, in the specific case of IgG, which can be present as a biotherapeutic substance in solution of a pharmaceutical preparation as a sample and is to be examined for aggregates. The fluorescent probe antibody in this case cannot bind a monomer bound to a capture molecule since its binding site is occupied by the capture molecule. Instead, it binds only to the monomer epitopes of the aggregates. This is thus quantifiable in the manner shown.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### REFERENCES

[1] Narhi, L. O., J. Schmit, et al. (2012). “Classification of protein aggregates.” J Pharm Sci 101(2): 493-498.
[2] Gamble, C. N. (1966). “The role of soluble aggregates in the primary immune response of mice to human gamma globulin.” Int Arch Allergy Appl Immunol 30(5): 446-455.
[3] Bachmann, M. F., U. H. Rohrer, et al. (1993). “The influence of antigen organization on B-cell responsiveness.” Science 262(5138): 1448-1451.
[4] Seong, S. Y. and P. Matzinger (2004). “Hydrophobicity: an ancient damage-associated molecular pattern that initiates innate immune responses.” Nat Rev Immunol 4(6): 469-478.
[5] den Engelsman, J., Garidel, P., et al. (2011). “Strategies for the Assessment of Protein Aggregates in Pharmaceutical Biotech Product Development.” Pharm. Res. 28: 920-933.

1. A method for detecting aggregates of biotherapeutic substances in a sample, comprising the following steps:
a. applying the sample to be examined onto a substrate,
b. adding probe molecules which are suitable for detection and which mark the aggregates of biotherapeutic substances by specifically binding thereto, and
c. detecting the marked aggregates of biotherapeutic substances, wherein step b) may be carried out before step a).

2. The method according to claim 1,
wherein
capture molecules for the aggregates are immobilized on the substrate before step a).

3. The method according to claim 1,
wherein
the sample is pretreated.

4. The method according to claim 1,
wherein
the substrate is made of glass.

5. The method according to claim 1,
wherein
the substrate is made of plastic.

6. The method according to claim 1,
wherein
the substrate has a hydrophilic coating.

7. The method according to claim 1,
wherein
the substrate is coated with dextran.

8. The method according to claim 1,
wherein
the substrate is coated with polyethylene glycol.

9. The method according to claim 7,
wherein
the dextran coating has a functionality for coupling biomolecules.

10. The method according to claim 8,
wherein
the polyethylene glycol coating has a functionality for coupling biomolecules.

11. The method according to claim 1,
wherein
the substrate is coated with a functionality for coupling biomolecules.

12. The method according to claim 6,
wherein
the hydrophilic coating is coated with a functionality for coupling biomolecules.

13. The method according to claim 2,
wherein
the capture molecules are bound to the substrate or to the coating.

14. The method according to claim 2,
wherein
the capture molecules are antibodies or fragments of antibodies.

15. The method according to claim 2,
wherein
the capture molecules are aptamers.

16. The method according to claim 2,
wherein
the capture molecules specifically bind one or more epitopes of the monomer of biotherapeutic substances.

17. The method according to claim 2,
wherein
the capture molecules specifically bind aggregates of biotherapeutic substances.

18. The method according to claim 1,
wherein
the probe molecules specifically bind one or more epitopes of the monomer of a biotherapeutic substance.

19. The method according to claim 1,
wherein
the probe molecules specifically bind aggregates of a biotherapeutic substance.

20. The method according to claim 1,
wherein
the probe molecules are marked with a detectable molecule.

21. The method according to claim 1,
wherein
the probe molecules are marked with fluorescent dyes.

22. The method according to claim 1,
wherein
one or more different probe molecules are used.

23. The method according to claim 1,
wherein
a mixture of various probe molecules with differently marked detectable molecules is used.

24. The method according to claim 1,
wherein
a mixture of identical probe molecules with differently marked detectable molecules is used.

25. The method according to claim 1,
wherein
detection is carried out by spatially resolving microscopy.

26. The method according to claim 1,
wherein
detection is carried out by spatially resolving fluorescence microscopy.

27. The method according to claim 1,
wherein
detection is carried out by confocal fluorescence microscopy, fluorescence correlation spectroscopy (FCS), optionally in combination with cross-correlation and single-particle-immunosolvent laser scanning assay, laser scanning microscopy (LSM), widefield microscopy and/or TIRF microscopy as well as the corresponding super-resolution variants STEP, SIM, STORM, dSTORM.

28. The method according to claim 1,
wherein
enough data points are collected during the detection that the detection of a single aggregate in front of the background signal is made possible.

29. The method according to claim 1,
wherein
an internal or external standard is used for quantifying and determining the size of aggregates of biotherapeutic substances.

30. The method according to claim 29,
wherein
the standard for quantifying and determining the size of aggregates of biotherapeutic substances consists of the monomers of the biotherapeutic substance.

31. The method according to claim 29,
wherein
the standard for quantifying and determining the size of aggregates of biotherapeutic substances consists of the monomers of the biotherapeutic substance and was covalently stabilized.

32. The method according to claim 29,
wherein
the standard for quantifying and determining the size of aggregates of biotherapeutic substances is a particle to which two or more identical or different polypeptide sequences are bound which are identical in sequence in the corresponding partial region of the sequences of the monomers of biotherapeutic substances bound by capture molecules and/or probe molecules.

33. The method according to claim 29,
wherein
the standard for quantifying and determining the size of aggregates of biotherapeutic substances is a particle to which two or more monomers of the biotherapeutic substance are bound.

34. The method according to claim 32,
wherein
the particle contains silica.

35. The method according to claim 32,
wherein
the particle has a hydrophilic coating.

36. A kit for the selective quantification of aggregates of biopharmaceutical agents by a method according to claim 1, the kit comprising one or more of the following components:
substrate;
probe molecules which bind to the aggregates of biotherapeutic substances by specific binding;
standard; and
capture molecule.
